(19) United States
(12) Patent Application Publication
(10) Pub. No.: US 2026/0295175 A1
(43) Pub. Date: Oct. 1, 2026

(54) NEEDLE LIGHT PIPE

(71) Applicant: Groton Consulting LLC, Derry, NH (US)

(72) Inventor: Christopher Petroff, Derry, NH (US)

(21) Appl. No.: 19/636,098

(22) Filed: Apr. 1, 2026

Related U.S. Application Data
(60) Provisional application No. 63/781,670, filed on Apr. 1, 2025.

(51) Int. Cl. A61M 5/32

(52) U.S. Cl. CPC A61M 5/3286; A61M 5/3287; A61M 2205/587

(57) ABSTRACT

Disclosed is a needle light pipe comprising a tubular needle body defining a lumen and a distal tissue-penetrating tip, and at least one optical path integrated with an outer diameter region of the tubular needle body and extending toward the distal tissue-penetrating tip. The at least one optical path is configured to direct light through a distal region of the tubular needle body for tissue interrogation, while the lumen remains unobstructed for fluid delivery or aspiration. Also disclosed are embodiments in which the optical path is formed by deposition, a graded-index profile, a step-index profile, or a channel receiving an optical member including a glass rod and glue having a different refractive index. In further instances, two optical paths include a transmission path and a reception path.

### CROSS REFERENCE TO RELATED APPLICATION

This application claims the benefit of U.S. Prov. Pat. App. No. 63/781,670, the entirety of which is hereby incorporated by reference.

### TECHNICAL FIELD

Embodiments described herein generally fall into the category of medical devices. In some embodiments, needle-based instruments are utilized, incorporating integrated optical pathways for tissue interrogation. In other embodiments, these instruments are designed to facilitate therapeutic delivery.

### DISCUSSION OF ART

Over time, the precise guidance of an introducer instrument into target tissues has proven to be highly significant in avoiding unintended trauma and ensuring proper therapeutic effect. Clinicians commonly rely on anatomical landmarks, ultrasound imaging, or fluoroscopic guidance to determine instrument placement. While these modalities provide useful information, they often add complexity, prolong procedural time, or introduce additional logistical requirements. As a result, there remains a continuing interest in enhancing real-time visualization directly at the working end of a needle-like device.

Fluoroscopic imaging is frequently employed in interventional settings to confirm the trajectory of slender instruments, but this technique exposes both the patient and the operator to ionizing radiation. Ultrasonography can provide real-time feedback without radiation; however, interpretation may be challenging in deeper or acoustically heterogeneous regions. Optical coherence tomography and near-infrared spectroscopy approaches have also been adapted for intraluminal visualization, yet the integration of these methods into devices has been hindered by size constraints and bulky external optical components that reduce maneuverability.

Existing designs have attempted to incorporate optical fibers within the central lumen of introducer devices to deliver and collect light for tissue interrogation. While these arrangements can yield detailed cross-sectional information, occupying the passageway precludes simultaneous fluid delivery or aspiration through the same channel. Removal of the fiber is sometimes proposed as a workaround, but this two-step process interrupts workflow and increases procedural duration. In certain cases, fibers are retracted after positioning, yet the resulting cavity can be difficult to seal in an effective fashion. Furthermore, non-disposable optical elements may require sterilization between uses, introducing potential risks of contamination. Configurations that rely on separate lumens for optics and therapy increase overall device diameter and may not be compatible with narrow anatomical access points. Accordingly, there is a need to address these shortcomings while preserving the functional capabilities of the instrument.

Alternative non-optical approaches have leveraged force sensors or pressure transducers integrated into the device housing to infer tissue boundaries based on penetration resistance. Such mechanical feedback mechanisms can provide useful directional cues, but they are often insensitive to variations in tissue optical properties that may indicate important anatomical structures. In addition, sensor calibration in viscous or fibrous tissues can be inconsistent, leading to ambiguous readings. The instrumentation required for pressure monitoring typically resides in a proximal hub, increasing system complexity. Signal processing algorithms are tasked with distinguishing between subtle changes in force, which can be confounded by patient movement or fluid flow. As a result, purely mechanical strategies have yet to achieve widespread adoption for fine-scale tissue differentiation.

Surface modifications of slender tools have been explored to enhance light reflection for visual feedback at the point of contact. For example, reflective coatings or engineered microstructures have been applied to proximal regions to scatter incident light and generate an image of adjacent tissues. Although these solutions can be fabricated using established coating processes, they often emit light in just one direction, limiting the field of view. The use of broad-area illumination also tends to increase device stiffness or thickness due to additional layers applied. Moreover, resolving fine tissue features requires careful alignment of illumination and detection optics, which is difficult to maintain during dynamic manipulation. As a result, many reflectance-based schemes fail to deliver consistent, high-resolution feedback in vivo.

In many minimally invasive interventions, instrument profiles are required to remain exceptionally small to gain access to confined anatomical spaces. Devices that incorporate multiple functional pathways for therapy and imaging often sacrifice portability and responsiveness. Bulky designs impede navigation in curvilinear or constrained regions, making them unsuitable for certain neurological, vascular, or joint applications. Accordingly, there is a persistent demand for devices that combine treatment capability with compact, integrated sensing features.

Strategies that require insertion and subsequent extraction of optical elements introduce procedural complexity and potential safety risks. The step of removing a fiber optic guide may leave a residual void that can permit fluid leakage or entrainment of air. Sealing mechanisms intended to occlude the lumen after fiber withdrawal can add manufacturing cost and may fail under physiological pressures. In addition, repeated insertion cycles may degrade sharpness or damage delicate tip geometries. There is also concern that non-disposable components may serve as reservoirs for biofilm formation or carryover contaminants. In situations demanding rapid device changes, the need for sterile, disposable optics further compounds logistical challenges. Thus, there is a need to minimize additional procedural steps without compromising tissue assessment.

Attempts to integrate coherent and incoherent light sources directly onto slender instruments have been reported in various contexts. Some embodiments use dual-path approaches to separate illumination and collection, seeking to optimize reflected signal fidelity. However, maintaining alignment between separate channels is technically demanding on sub-millimeter scales. The incorporation of graded-index materials has been proposed to confine light propagation paths, yet deposition techniques for such profiles often require specialized equipment and are not widely adopted in disposable device manufacturing. Similarly, step-index schemes can reduce modal dispersion but may still struggle with mode-dependent attenuation over short path lengths. These factors have limited the translation of advanced optical schemes to commercially viable products, leaving many devices as research prototypes rather than clinical tools.

Applying thin, uniform optical coatings to curved metal surfaces poses a distinct set of manufacturing challenges. Variations in thickness can lead to inconsistent light transmission or unintended scattering. Achieving repeatable performance across batches remains difficult for numerous conventional deposition methods.

Clinicians increasingly seek single-use instruments that seamlessly combine diagnostic sensing and therapeutic delivery. Such devices are designed to support fluid injection or aspiration while providing immediate feedback on local tissue characteristics. Ideally, sensing elements are configured to avoid obstructing the flow path or compromising structural integrity. Rapid device exchange under sterile conditions is often required in busy procedural settings. Any added optical components are expected to be both robust and cost-effective. Developing an approach that meets these criteria has remained a significant challenge.

Reuse of non-disposable optical components raises concerns about sterility and cross-contamination. While sterilization protocols exist, they can degrade optical performance over time. The potential for residual biological material underscores the need for disposable or permanently integrated solutions.

Devices that incorporate removable elements often require additional sealing assemblies to maintain a sterile barrier. Designing reliable seals for millimeter-scale openings introduces material compatibility and durability concerns. In some configurations, seals are designed to tolerate repeated pressurization without leakage. These engineering demands increase manufacturing complexity and cost.

Implementing multiple sensing channels on a miniature platform complicates both assembly and signal routing. Channel-to-channel crosstalk can reduce measurement accuracy, particularly in reflective or scattering tissues. Isolation layers and shielding increase dimensional requirements and may affect mechanical properties. Balancing the number of channels with overall instrument size has proven difficult. There remains a need to optimize signal integrity without expanding the profile.

Proximal connectors and light delivery consoles often account for a significant portion of the device footprint. Large connectors can interfere with simultaneous use of ancillary equipment. Systems designed for high-resolution imaging typically require benchtop modules that are incompatible with bedside workflows. Thus, reducing the bulk of these attachments is important for facilitating point-of-care adoption.

Fabrication processes that involve multiple deposition, machining, and polishing steps can be both time-consuming and costly. Precision alignment of micro-scale features demands tight tolerances and complex fixtures. Post-assembly calibration of optical pathways may be required to ensure consistent performance. Each additional manufacturing stage increases the risk of defects and reduces overall throughput. Scaling such processes to high-volume production remains a formidable task. Furthermore, integrating disposability constraints adds further design considerations. Addressing these manufacturing challenges is necessary to deliver economically feasible solutions.

Streamlining device assembly by reducing the number of discrete components can improve reliability and lower costs. Integrating functional pathways directly into the structural substrate holds promise in this regard. However, material choices that support both mechanical strength and optical functionality are limited. Achieving a balance between durability and performance is important.

Interventional instruments are required to withstand bending, torsion, and repeated insertions without degradation. Delicate optical features are particularly vulnerable to mechanical stresses. Protective layers or encapsulation can enhance durability but may impact optical efficiency. As a result, the development of resilient sensing elements is an important consideration.

Recent trends in minimally invasive therapies have driven demand for ever-smaller, high-resolution sensing tools. Micro-fabrication techniques have enabled novel optical structures on planar substrates. Translating these approaches to cylindrical or tapered geometries common in clinical instruments remains challenging. Efforts to deposit graded or step index profiles on curved surfaces are underway, but uniformity and reproducibility issues persist. Overcoming these obstacles would significantly advance the state of the art.

Many commercially available instruments still rely on single-mode or multimode fibers affixed externally to the device shaft. Such configurations are ill-suited for applications requiring precise lumen patency. The lack of integrated sensing pathways constrains real-time feedback capabilities.

In view of the foregoing, there remains a need for an instrument capable of providing localized tissue interrogation while preserving an unobstructed working channel. The ideal solution would integrate illumination and collection pathways in a low-profile arrangement without compromising mechanical or fluidic performance. Such an instrument is expected to be suitable for scalable fabrication and disposable use. Addressing these requirements would advance minimally invasive diagnostics and therapeutics.

### SUMMARY

This current disclosure overcomes the above outlined limitations by providing a very low-profile method of interrogating vessels. One or two light paths are used although more are possible. With one light path, the reflected light is returned via the same path the light is delivered by. With two or more paths light is delivered by at least one path and collected from another location by at least one other path.

In certain embodiments, the light delivered may be incoherent or coherent. If coherent light is used, OCT can be implemented with forward viewing to determine the tissue features in the direction of travel. Coherent light may also be used to do determine the tissue type through near-infrared spectroscopy (NIRS technology). Incoherent light may also be used to type tissues. The incoherent light could vary in intensity or wavelength with time, so the reflectivity of the tissue in front of the needle at different intensities and wavelengths is measured.

In some embodiments, a needle light pipe includes a tubular needle body defining a lumen and a distal tissue-penetrating tip, and at least one optical path integrated with an outer diameter region of the tubular needle body and extending toward the distal tissue-penetrating tip. The at least one optical path may be configured to direct light through a distal region of the tubular needle body for tissue interrogation, while the lumen remains unobstructed by the at least one optical path and is configured for fluid delivery or aspiration.

In some embodiments, the at least one optical path may be formed by deposition on the outer diameter region of the tubular needle body and may include a graded-index profile or a step-index profile. In some embodiments, the tubular needle body may define a channel in which the at least one optical path is received. For example, the at least one optical path may include a glass rod disposed in the channel, and glue having a refractive index different from that of the glass rod may be disposed in the channel. In some embodiments, the at least one optical path may include two optical paths, including a transmission path and a reception path. The transmission path and the reception path may be spaced from each other by about 5 degrees to 180 degrees about a central axis of the tubular needle body. In some embodiments, a distal end of the at least one optical path may be positioned from about 0 mm to about 1 mm proximal to the distal tissue-penetrating tip. In some embodiments, the outer diameter of the tubular needle body may include an opaque coating positioned to constrain light to the at least one optical path. In some embodiments, a distal cut face of the tubular needle body may be polished to improve light transmission through the at least one optical path. In some embodiments, a proximal end of the at least one optical path may be optically coupled to a connector including a precision glass tube.

In some embodiments, a method includes advancing a tubular needle body defining a lumen and a distal tissue-penetrating tip toward tissue, the tubular needle body having at least one optical path integrated with an outer diameter region of the tubular needle body and extending toward the distal tissue-penetrating tip, directing light through a distal region of the tubular needle body via the at least one optical path to interrogate the tissue, and delivering fluid through the lumen or aspirating fluid through the lumen while the lumen remains unobstructed by the at least one optical path.

In some embodiments, a method of constructing a needle light pipe includes providing a tubular needle body defining a lumen, forming at least one channel in an outer diameter region of the tubular needle body, the at least one channel extending toward a distal region of the tubular needle body, positioning an optical member in the at least one channel, disposing a glue in the at least one channel around the optical member, the glue having a refractive index different from a refractive index of the optical member such that the optical member and the glue define at least one optical path integrated with the outer diameter region of the tubular needle body, hardening the glue, forming a distal tissue-penetrating tip on the tubular needle body, and polishing a distal face of the tubular needle body to improve light transmission through the at least one optical path, wherein the lumen remains unobstructed by the at least one optical path. In some embodiments, the method may further include placing a polytetrafluoroethylene (PTFE) shrink wrap over the tubular needle body before hardening the glue and removing the PTFE shrink wrap after hardening the glue to provide a smooth outer diameter. In some embodiments, the method may further include coating the outer diameter region of the tubular needle body with an opaque material after hardening the glue, wherein the opaque material may include silver or gold. In some embodiments, forming the distal tissue-penetrating tip may include angle cutting the tubular needle body.

### BRIEF DESCRIPTION OF THE DRAWINGS

Features and advantages of embodiments of the present invention will become apparent on reading the detailed description below with reference to the drawings, which are illustrative but non-limiting, wherein:

FIG. 1 illustrates an example embodiment of a needle having one or more optical light paths formed along an outer diameter of the needle.

FIG. 2 illustrates light being channeled in an optical light path.

FIG. 3 illustrates an example graded-index profile deposited on an outside surface of a tube or needle.

FIG. 4 illustrates an example embodiment in which one or more channels formed in or around a metal tube to define one or more light-transmission paths.

FIG. 5 illustrates an example attachment an external light path to one or more needle light channels.

FIG. 6 illustrates an example cross-section optical coupling configuration between a fiber and a disposable light-channel structure.

FIG. 7 illustrates an example embodiment in which transmission and reception paths are embedded in grooves in the outer diameter of a needle.

FIG. 8 illustrates an end view of a needle showing one or more light-transmission paths formed in the metal tube and receiving optical members therein.

FIG. 9 illustrates an example needle outer diameter after removal of a shrink-wrap layer, resulting in a smooth outer surface.

FIG. 10 illustrates an example angled needle tip after polishing of a cut face to improve light transmission and needle sharpness.

FIG. 11 illustrates an example connector arrangement for coupling a light path to a console, including a precision glass tube and an angled physical contact connector or spherical glue interface.

### DETAILED DESCRIPTION

### Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

An element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features.

Moreover, unless explicitly stated to the contrary, the transitional phrases “comprising,” “including,” or “having” are open ended and can include additional elements not recited in the claim. Claims that use these open-ended transitional phrases also include intermediate transitional phrases, e.g. “consisting essentially of,” and close-ended transitional phrases, e.g. “consisting of.”

As used herein, ranges and amounts can be expressed as “about” a particular value or range. About also includes the exact amount. Hence “about 5 μL” means “about 5 μL” and, also “5 μL.” Generally, the term “about” includes an amount that would be expected to be within experimental error or within the error expected from manufacturing, production, or experimental tolerances.

Suitable alterations to the above are readily apparent to those of skill in the art and naturally are encompassed and expressly contemplated. For example, normal manufacturing tolerances may induce variances from the above presented formulations without departing from the broader scope of this invention.

Section headings are provided for convenience of reference only and are not intended to limit the scope of the present disclosure in any way. Subject matter described in one section may be used alone or in combination with subject matter described in one or more other sections, unless the context clearly indicates otherwise. Accordingly, the organization of the present disclosure under particular headings should not be construed as requiring that any embodiment, feature, or aspect be limited to the specific section in which it is described.

### ABBREVIATIONS

APC—angled physical contact
NIRS—near-infrared spectroscopy
OCT—optical coherence tomography
OD—outer diameter
PTFE—polytetrafluoroethylene

In the following detailed description, example embodiments of a needle-based instrument having one or more optical light paths integrated along an outer diameter of a needle are described with reference to FIGS. 1-11. The disclosed configurations may enable forward tissue interrogation while preserving a central lumen for fluid delivery, aspiration, or other therapeutic use. As discussed below, the light path or light paths may be formed by deposition, masking, machining, groove formation, or placement of glass or plastic optical members in channels formed in the needle body and may be configured as single-path or dual-path arrangements for light delivery and light collection. Various embodiments may further employ incoherent light, coherent light, graded-index structures, step-index structures, reflective or opaque coatings, polished or cleaved optical interfaces, and proximal coupling features configured to connect the needle to an external console while maintaining a low-profile, durable, and manufacturable structure.

An example embodiment is shown in FIG. 1. A light path (one or more) 100 is created on a light path outer surface 102 of needle 104 by a deposition process.

The light path 100 does not exit at the very end of the needle so as not to disturb the sharp point 106. One or two light paths 100 can be used. If two light paths are used, they can be on the same side of the sharp point 106 or different sides.

The deposition process can vary depending upon the signal that is being transmitted. For the simple transmission of incoherent light, the light path 100 may be made simply by dipping the needle 104 in glue to create an annulus for transmitting light. The annulus would be plated by sputter coating or another method to prevent light from leaking from the light path outer surface 102. The needle 104 is composed of a tube made of stainless steel or another suitable metal. After coating the tube of the needle 104, the end 108 is finished. A secondary operation may be performed to shape the surface of the exit/entry of the light path 110 into the tissue.

The light path 100 may encompass the entire circumference of the needle 104, or it may by constrained to certain paths as shown in FIG. 1. If the entire circumference is used for some transparent tissues, visualization of the tissue may be done directly on the exterior of the body, by looking at how much light is returned.

To constrain the light path 100 to certain parts of the circumference, the light path 100 may be removed mechanically after application. Alternatively, the tube of the needle 104 may be masked before light path 100 application and the masking removed to only leave sections of the light path 100 application.

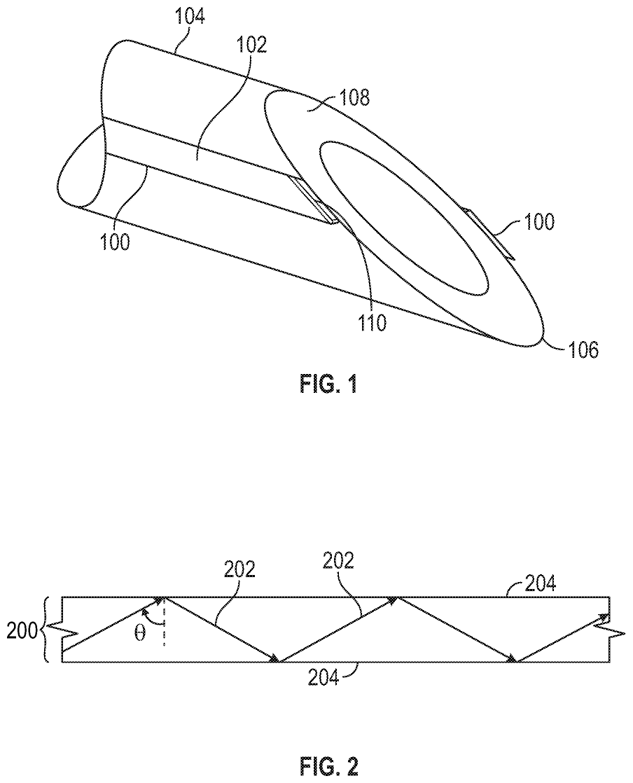

FIG. 2 illustrates light being channeled in an optical light path. A single index layer of optically transparent material 200 (e.g., glass or clear plastic) deposited on the surface of a needle has the disadvantage that light can take a plurality paths 202. A single central path has the shortest arrival distance, but off axis light may bounce off the outer coating 204 and the metal needle to create a longer path. Additionally, light paths 204 may also go around the circumference of the fiber. Especially with coherent light transmission, these different path lengths may make it difficult to distinguish between tissues being interrogated. For certain tissue interrogation applications, this path length difference may be acceptable, and this lower cost method of light channel construction may be the optimum design.

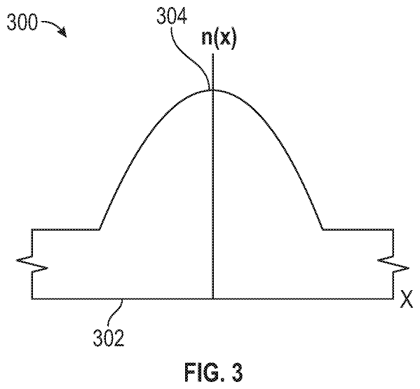

Alternatively, a fiber with a 2D graded index profile 300 may be deposited on the outside of a tube 302. A graded index profile has a higher index in the center of the fiber 304 as shown in FIG. 3. The higher index focuses the light to the center of the fiber.

2D graded index profiles have been manufactured by deposition process (see, Fabrication and characterization of a planar gradient-index, plasma-enhanced chemical vapor deposition lens, 1 Oct. 1997 y Vol. 36, No. 28 y APPLIED OPTICS, the entirety of which is incorporated herein by reference) with a thickness of only a few microns thick. This method improves light transmission and is useful in constraining path lengths.

The graded index profile may be selected to allow all modes to have the same group velocity, reducing dispersion. The higher modes speed up as they go away from the center of the optical path 304, compensating for their longer path lengths.

In alternative embodiments, a step index deposition process may be used instead of a graded index process. The discontinuity in index at the step is useful for multimode transmission.

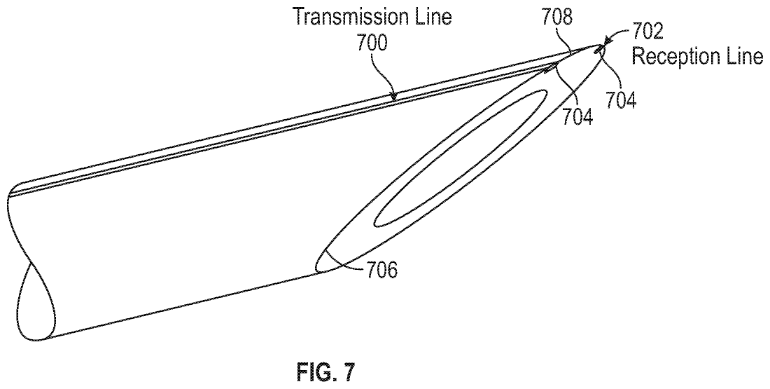

A 2D index profile may also be deposited onto a needle with channel(s) in the metal tube. As shown in further detail in FIGS. 7-10, the deposition outside of the tube is removed, leaving a channel for transmission of light.

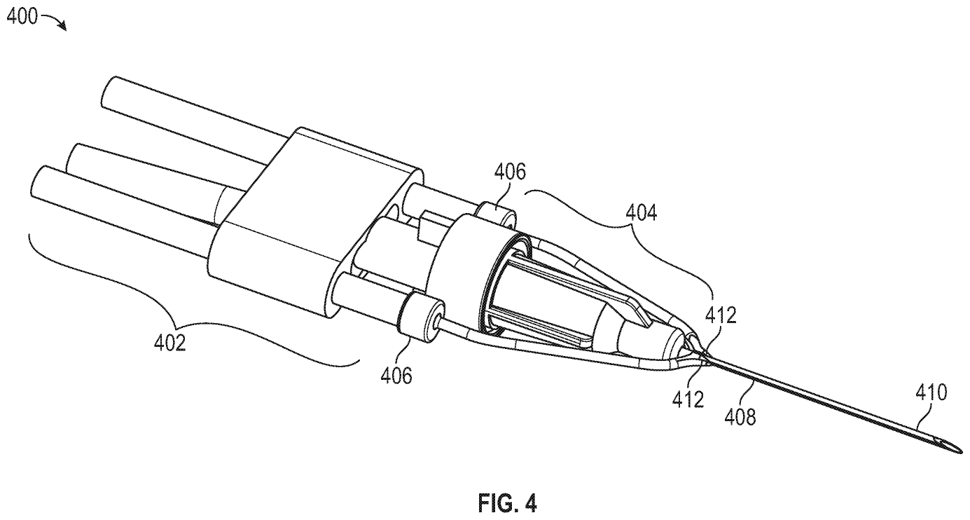

FIG. 4 further illustrates a possible attachment of the light channels to a console 400. A rear non-disposable portion 402 connects to a disposable front mount 404 bearing light source attachment points 406 which engage with light paths/channels 408 integrated with needle 410.

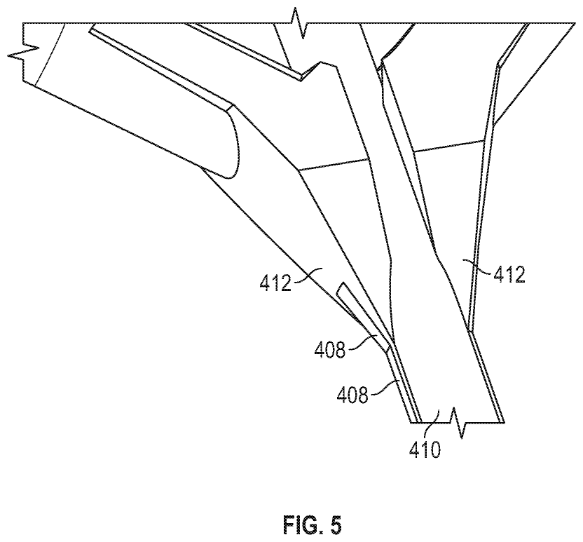

Seen in FIG. 5, deposition of light channels 408 over plastic take offs 412 will allow efficient light transmission down needle 410.

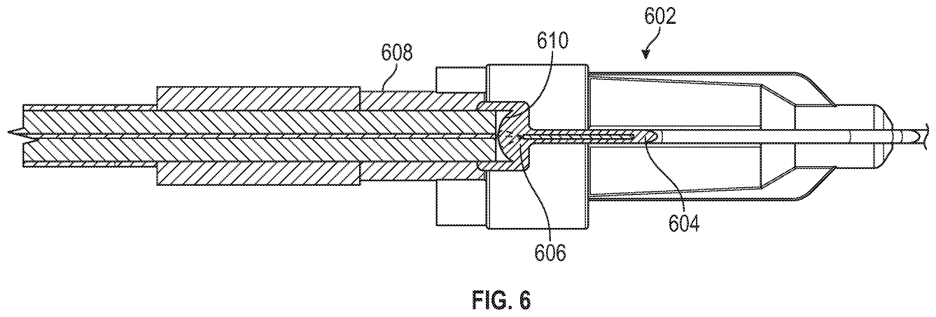

FIG. 6 illustrates an example cross-section optical coupling configuration between a fiber and a disposable light-channel structure. The disposable portion 602 contains a single mode fiber 604 (progressively cut-away in layers from right to left). The end of the fiber 606 may be cleaved at 0° or it may be cleaved at angles of 0°-15° with a typical cleave angle of 8°. This angle reduces reflection, but other angles may be used such as 10°. No angle is required if the imaging technique is not sensitive to back reflections. The non-disposable front mount 608 may also contain a single mode fiber and face a disposable domed surface 610 integrated with the disposable portion 602 to allow light to travel into it.

When a single mode fiber is not used or any other path where dispersion may occur, the length of the paths beyond the fiber are kept short, so dispersion & attenuation are kept low. By way of example, a needle length of 20 mm is useful for procedures near the skin. A needle length of 100 mm is useful for spinal and neck procedures

FIG. 7 provides an example embodiment. In this embodiment transmission line 700 and reception paths 702 are embedded in grooves 704 in the outer diameter 706 of needle 708. This embodiment has a small size, which allows the paths 700/702 to be located closer to the tip of the needle 708.

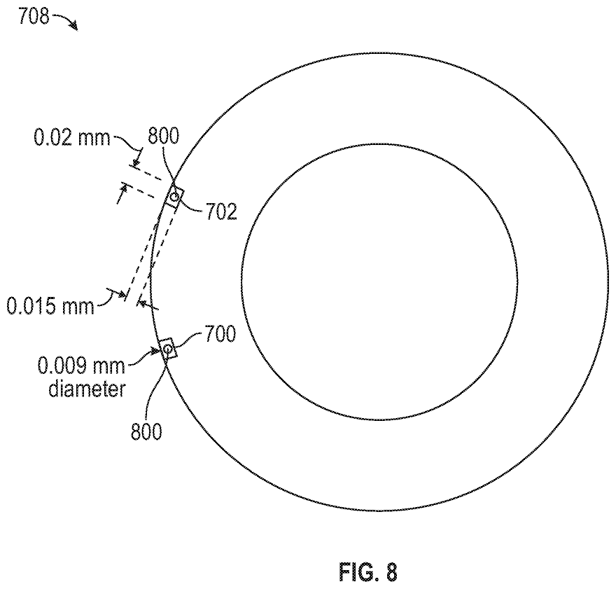

FIG. 8 shows an end view of needle 708. The transmission path(s) 700/702 are extruded or machined into the metal tube composing the body of needle 708. In an example embodiment the paths 700/702 are 0.015 mm deep and 0.02 mm wide slots. A 0.009 mm diameter glass rod 800 is fitted into the slot and the slot is filled with glue. The glue has a different index of refraction than the glass rod, thus the glue+glass rod will act as a single mode fiber. Suitable glues include optical glues such as Norland™ 132.

Plastic rod may be used as an alternative to the glass. Shrink wrap, such as PTFE shrink wrap, is placed over the tube and the glue is hardened.

The transmission/receiving paths 700/702 of the light with respect to the central axis of the needle are shown 40° from each other, on different sides of the point (see, FIG. 7 for further detail). The paths 700/702 may be placed such that they are spaced for the desired interrogation length of the tissue. If the light paths 700/702 are further apart crosstalk will be reduced. Paths can be 5° to each other for a maximum amount of returned light, thus the strongest signal.

The slot width and depth and the glass rod/fiber diameter may be varied to accommodate different frequencies to be transmitted or received. In certain embodiments, a glass diameter of 0.05 mm may be used for multimode transmission. If a dye or tracer in the tissue is being interrogated the dimensions may be optimized to pick that up. In certain embodiments, multiple paths of varying diameters may be created in order to accommodate one or more ranges of light wavelengths.

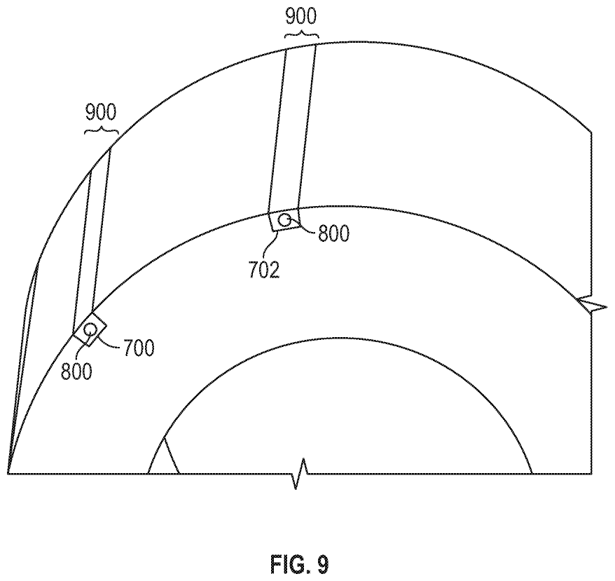

In certain embodiments, glue may be used to pot the glass fiber and/or rod, creating a light channel around it. The glue is confined by shrink wrap. Once the glue is hardened, the shrink wrap is pulled off the tube, resulting in a smooth, atraumatic outer diameter 900 as shown in FIG. 9.

After the glue has hardened, the outer diameter of the needle may be coated with an opaque material to constrain the light to the channel. Acceptable coatings include silver and gold. Sputter coating or dipping may be used to apply them.

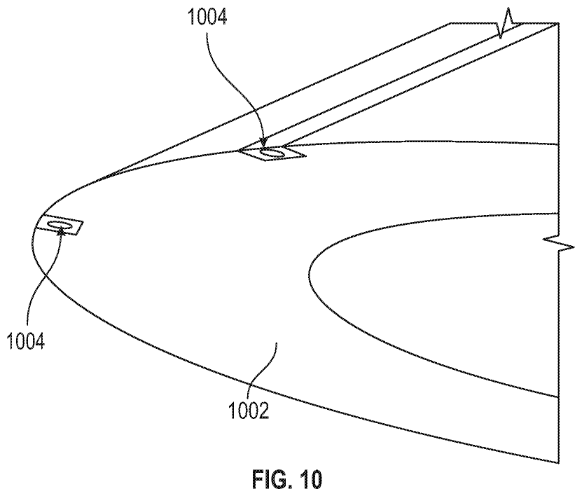

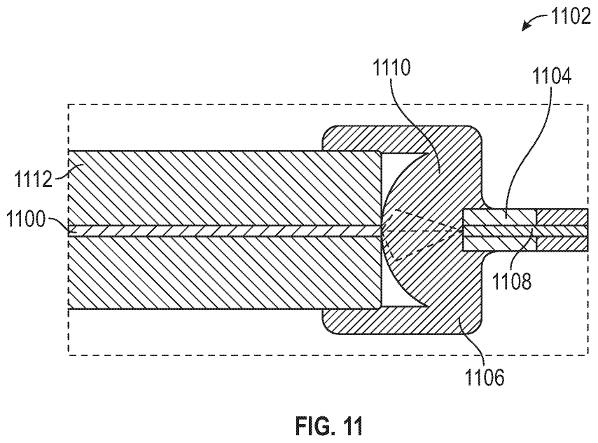

Then, if not cut already, the metal tube may be angle cut to create the sharp point. As shown in FIG. 10, then the entire cut face 1002 is polished, which creates a smooth surface on the glass faces 1004, improving the light transmission. This polishing can also remove any burr created from cutting the tube, improving the sharpness of the needle.

Since the end of the glass is right at the face of the needle, it will tend to remain clean as the needle is manipulated. Being embedded in glass, it is securely attached to the needle.

In some embodiments, to keep the light more axial to the needle and improve durability, a glass end is cleaved and kept slightly (0-1 mm) proximal to an angled tip (e.g., the light path end is set back from the face of the needle). The cleave may be angled at 8° or 10° to reduce reflections although angles from 0°-15° are possible. In this case, the light will transmit through the glue and into the body. The glue will act as the optical surface in contact with the body. Alternatively, glue may not be present and the cleaved glass face is in contact with the body.

To attach to a console, a glass fiber may be laid in a slot in a needle hub, which is also filled with glue and coated to create a light channel.

FIG. 11 illustrates an example connector arrangement for coupling a non-disposable light path 1100 to a console 1102, including a precision glass tube 1104 and an angled physical contact connector or spherical glue interface. The connector 1106 may be fitted with a precision glass tube 1104 to center the glass rod 1108 which is inserted through it. The glass rod and glass tube may be polished to create an angled, physical contact (APC) connector or it may be embedded in a spherical glue surface 1110. In certain embodiments the light path 1100 may be embedded in a glass or plastic fiber and/or rod 1112.

### EXAMPLES

In one example embodiment, a needle light pipe includes a tubular needle body defining a lumen and a distal tissue-penetrating tip, and at least one optical path integrated with an outer diameter region of the tubular needle body and extending toward the distal tissue-penetrating tip. The at least one optical path may be configured to direct light through a distal region of the tubular needle body for tissue interrogation, while the lumen remains unobstructed by the at least one optical path and remains available for fluid delivery or aspiration. In some implementations, the at least one optical path may be formed by deposition on the outer diameter region of the tubular needle body and may have a graded-index profile or a step-index profile. In some implementations, the tubular needle body may define a channel receiving the at least one optical path, such as a glass rod disposed in the channel with a glue having a refractive index different from that of the glass rod. In some embodiments, two optical paths may be provided, including a transmission path and a reception path spaced from each other by about 5 degrees to about 40 degrees about a central axis of the tubular needle body. A distal end of the at least one optical path may be positioned from about 0 mm to about 1 mm proximal to the distal tissue-penetrating tip. The outer diameter of the tubular needle body may further include an opaque coating configured to constrain light to the at least one optical path, a distal cut face may be polished to improve light transmission through the at least one optical path, and a proximal end of the at least one optical path may be optically coupled to a connector including a precision glass tube.

In another example embodiment, a method of using a needle light pipe includes advancing a tubular needle body defining a lumen and a distal tissue-penetrating tip toward tissue, wherein the tubular needle body has at least one optical path integrated with an outer diameter region of the tubular needle body and extending toward the distal tissue-penetrating tip. Light may be directed through a distal region of the tubular needle body via the at least one optical path to interrogate the tissue during insertion, positioning, or treatment. While optical interrogation is performed, fluid may be delivered through the lumen or fluid may be aspirated through the lumen, with the lumen remaining unobstructed by the at least one optical path. In this manner, the needle may provide both optical functionality and fluid-based functionality in a single instrument without requiring the optical path to occupy the lumen.

In a further example embodiment, a method of constructing a needle light pipe includes providing a tubular needle body defining a lumen, forming at least one channel in an outer diameter region of the tubular needle body with the at least one channel extending toward a distal region of the tubular needle body, positioning an optical member in the at least one channel, and disposing a glue in the at least one channel around the optical member. The glue may have a refractive index different from a refractive index of the optical member such that the optical member and the glue define at least one optical path integrated with the outer diameter region of the tubular needle body. The method may further include hardening the glue, forming a distal tissue-penetrating tip on the tubular needle body, and polishing a distal face of the tubular needle body to improve light transmission through the at least one optical path, while the lumen remains unobstructed by the at least one optical path. In some implementations, a PTFE shrink wrap may be placed over the tubular needle body before hardening the glue and removed after hardening the glue to provide a smooth outer diameter. In some implementations, the outer diameter region of the tubular needle body may be coated with an opaque material after hardening the glue, and the opaque material may include silver or gold. In some implementations, forming the distal tissue-penetrating tip may include angle cutting the tubular needle body.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A needle light pipe comprising:
a tubular needle body defining a lumen and a distal tissue-penetrating tip; and,
at least one optical path integrated with an outer diameter region of the tubular needle body and extending toward the distal tissue-penetrating tip;
wherein the at least one optical path is configured to direct light through a distal region of the tubular needle body for tissue interrogation;
wherein the lumen is unobstructed by the at least one optical path and is configured for fluid delivery or aspiration.

2. The needle light pipe of claim 1, wherein the at least one optical path is formed by deposition on the outer diameter region of the tubular needle body.

3. The needle light pipe of claim 1, wherein the at least one optical path comprises a graded-index profile on the outer diameter region of the tubular needle body.

4. The needle light pipe of claim 1, wherein the at least one optical path comprises a step-index profile on the outer diameter region of the tubular needle body.

5. The needle light pipe of claim 1, wherein the tubular needle body defines a channel in which the at least one optical path is received.

6. The needle light pipe of claim 5, wherein the at least one optical path comprises a glass rod disposed in the channel.

7. The needle light pipe of claim 6, wherein glue having a refractive index different from that of the glass rod is disposed in the channel.

8. The needle light pipe of claim 1, wherein the at least one optical path comprises two optical paths including a transmission path and a reception path.

9. The needle light pipe of claim 8, wherein the transmission path and the reception path are spaced from each other by 5 degrees to 180 degrees about a central axis of the tubular needle body.

10. The needle light pipe of claim 1, wherein a distal end of the at least one optical path is positioned 0 mm to 1 mm proximal to the distal tissue-penetrating tip.

11. The needle light pipe of claim 1, wherein an outer diameter of the tubular needle body includes an opaque coating positioned to constrain light to the at least one optical path.

12. The needle light pipe of claim 1, wherein a distal cut face of the tubular needle body is polished to improve light transmission through the at least one optical path.

13. The needle light pipe of claim 1, wherein a proximal end of the at least one optical path is optically coupled to a connector comprising a precision glass tube.

14. A method comprising:
advancing a tubular needle body defining a lumen and a distal tissue-penetrating tip toward tissue, the tubular needle body having at least one optical path integrated with an outer diameter region of the tubular needle body and extending toward the distal tissue-penetrating tip;
directing light through a distal region of the tubular needle body via the at least one optical path to interrogate the tissue; and
delivering fluid through the lumen or aspirating fluid through the lumen while the lumen remains unobstructed by the at least one optical path.

15. A method of constructing a needle light pipe, comprising:
providing a tubular needle body defining a lumen;
forming at least one channel in an outer diameter region of the tubular needle body, the at least one channel extending toward a distal region of the tubular needle body;
positioning an optical member in the at least one channel;
disposing a glue in the at least one channel around the optical member, the glue having a refractive index different from a refractive index of the optical member, such that the optical member and the glue define at least one optical path integrated with the outer diameter region of the tubular needle body;
hardening the glue;
forming a distal tissue-penetrating tip on the tubular needle body; and
polishing a distal face of the tubular needle body to improve light transmission through the at least one optical path,
wherein the lumen remains unobstructed by the at least one optical path.

16. The method of claim 15, further comprising placing a PTFE shrink wrap over the tubular needle body before hardening the glue.

17. The method of claim 16, further comprising removing the PTFE shrink wrap after hardening the glue to provide a smooth outer diameter.

18. The method of claim 15, further comprising coating the outer diameter region of the tubular needle body with an opaque material after hardening the glue.

19. The method of claim 18, wherein the opaque material comprises silver or gold.

20. The method of claim 15, wherein forming the distal tissue-penetrating tip comprises angle cutting the tubular needle body.
